(19) United States
(12) Patent Application Publication
(10) Pub. No.: US 2019/0009111 A1
(43) Pub. Date: Jan. 10, 2019

(54) NON-INVASIVE AND OPTIMIZED SYSTEM FOR THE REJUVENATION AND REMOVAL OF WRINKLES OF THE SKIN

(71) Applicants: Gunnar MYHR, Oslo (NO); Bjørn A. J. Angelsen, Trondheim (NO)

(72) Inventors: Gunnar MYHR, Oslo (NO); Bjørn A. J. Angelsen, Trondheim (NO)

(21) Appl. No.: 16/003,549

(22) Filed: Jun. 8, 2018

(30) Foreign Application Priority Data
Jun. 8, 2017 (DK) PA2017 00339

(51) Int. Cl. A61N 7/02

(52) U.S. Cl. CPC A61N 7/02; A61N 2007/0082

(57) ABSTRACT

The invention relates to a system and method for the removal of wrinkles and/or provide the rejuvenation of the human skin by use of ultrasound. The method comprises determining a 3D image of a region of the skin using ultrasound, determining a focal depth of the ultrasonic beam for different locations of the skin based on the 3D image, performing the treatment by heating the skin at different locations using an ultrasonic beam, and adjusting the focal depth of the ultrasonic beam according to the determined focal depths during the process of heating the skin at different locations.

### CROSS-REFERENCE TO RELATED APPLICATIONS

This application claims the priority of Danish patent application no. DK 2017 00339, filed Jun. 8, 2017, which application is incorporated herein by reference.

### BACKGROUND OF THE INVENTION

This is an invention related to systems and optimized procedures for non-invasive rejuvenation of the human skin by providing heat deposits or coagulations within the skin by the application of ultrasound.

### 1. The Structure of the Human Skin

FIG. 1 shows the various primary layers of the human skin, the epidermis and the dermis subgroups. The location of the SMAS region is also stated, along with locations for applying heat deposits in rejuvenation applications.

The human skin has up to seven layers of ectodermal tissue and guards the underlying muscles, bones, ligaments and internal organs.

The term “skin” or “human skin” represents all layers of ectodermal tissue or layers, including subcutaneous fat layers and the SMAS layer(s) and/or skin descriptions described in this document, including figures.

The human skin is the largest organ of the body, approximately 1.75 m². Its main purpose is to protect and conceal the total organism. The skin constitutes of several layers. The epidermis is the outermost layer. The dermis is the layer of skin beneath the epidermis. It consists of connective tissues and cushions the body from stress and strain. The dermis provides tensile strength and elasticity to the skin through an extracellular matrix composed of collagen fibrils, micro fibrils, and elastic fibers.

The superficial muscular aponeurotic system (SMAS) is an area of and adjacent to musculature of the face. The SMAS lies deep and underneath to the subcutaneous fat. It envelops the muscles of facial expression and extends superficially to connect with the dermis. The SMAS layer is composed of collagen and elastic fibers similar to the dermal layer of the skin. The SMAS is the target of and is manipulated during facial cosmetic surgery, especially rhytidectomy (face lift).

The SMAS can also be a desirable target for non-invasive skin tightening procedures, like High Intensity Focus Ultrasound (HIFU), SeminCutan Med Surg. 2013 March; 32(1):18-25.

However, with non-optimal HIFU systems and procedures, one can miss the SMAS layer(s), and destroy the subcutaneous fat layers, thus providing more severe wrinkles.

### 1.1 Published Human Skin Thickness Data Varies.

In http://emedicine.medscape.com/article/1294744-overview states, it is stated a general norm, that skin is thickest on the palms and soles of the feet at 1500 μm (1.5 mm), while the thinnest skin is found on the eyelids and in the post auricular region at 50 μm (0.05 mm).

In AesthetSurg J. 2015 November; 35(8):1007-13 full-thickness punch biopsy samples were obtained at 39 predetermined anatomic locations of the face from 10 human cadaveric heads. The area of the face with the thickest dermis was the lower nasal sidewall (1969.2 μm, dRT: 2.59), and the thinnest was the upper medial eyelid (758.9 μm, dRT: 1.00). The area with the thickest epidermis was the upper lip (62.6 μm, eRT: 2.12), and the thinnest was the posterior auricular skin (29.6 μm, eRT: 1.00).

With reference to FIGS. 1 and 2 and the above stated publications, the practical skin thickness (epidermis+dermis) in the face would be between 0.5 mm and 1.2 mm. Underneath the subcutaneous fat is where the SMAS layer is located. Typical thickness of the SMAS layer is 0.4 mm.

After the age of 20, a person produces about 1% less collagen in the skin each year. As a result, the skin becomes thinner and more fragile with age. There is also diminished functioning of the sweat and oil glands, less elastin production, and less glycosaminoglycans or GAGs (which keep the skin hydrated), https://www.scientificamerican.com/article/why-does-skin-wrinkle-wit/.

The first signs of ageing start when the collagen production starts to decline, firstly manifested by fine lines and wrinkles at the outer corners of the eyes. Later, deeper wrinkles form between the nose and mouth. A face appears aged when the local skin has undergone change; it has suffered loss of volume, experienced reduced elasticity and been subject to lipo (fat) atrophy, AesthetSurg J. 2014 September; 34(7):1099-110.

Exposure to the Sun, skin disorders, aging and heredity can all contribute to skin irregularities on the face and elsewhere on the body. These include textural irregularities, acne scars, pigmentation changes like freckles, sunspots, age spots or visible blood vessels. In addition, skin may lose tone, feel less firm and lose the healthy glow that is evident in younger skin. All these factors are or can be associated with aging and/or provide the appearance of wrinkles.

The following skin conditions, but not limited to, are included in the concept of wrinkles, and can be addressed by the treatment procedures outlined in this document:
Static wrinkles: These wrinkles are visible at all times and do not change in appearance with facial movements
Dynamic wrinkles: These are expression lines that may appear as folds when the skin is not moving, and deepen with facial movements or expressions
Pigmentation: Freckles, sun spots, or other darkened patches of skin result mainly from sun exposure
Scars: As the result of acne or injury to the skin, scars may be rolling (a wavy appearance to the skin), pitted, discolored, or have raised borders
Vascular conditions: Blood vessels visible on the surface of the skin, vascular lesions that appear as tiny blood-filled blisters or even a constant flush of facial redness
Loss of skin tone: Weakening of the supportive skin structures (collagen and elastin fibers) that result in a loss of skin firmness or the development of cellulite
Dull skin: Skin that has lost the vibrant glow from a buildup of dead skin cells and clogged pores.

https://www.plasticsurgery.org/cosmetic-procedures/skin-rejuvenation-and-resurfacing

Wrinkle removal and rejuvenation are synonymous concepts in this document.

### 2. Non-Invasive Remodeling Techniques of the Skin

Facial and neck skin remodeling has traditionally been addressed using surgical (face) lifting procedures. Later, non-surgical procedures were developed with the utilization and application of radiofrequency (RF) and ablative lasers. The mode of operandi for these techniques is the application and deposit of heat. However, these procedures produced e.g. inconsistent clinical results, extensive postoperative recovery requirements and the risk of delayed dyspigmentation, Arch Dermatol. 1999; 135:444-454, SeminCutan Med Surg. 2013 March; 32(1):18-25.

Non-invasive techniques have developed with the aim of inducing thermal injury within the dermis, without epidermal damage, thus avoiding potential complications due to the procedures, Journal of Cosmetic and Laser Therapy, 2015; 17: 230-236. Some non-ablative rejuvenation (NAR) devices use intense pulsed light, pulsed-dye lasers and radiofrequency (RF). Monopolar RF therapy delivers uniform heat at a controlled depth in the dermal layers, Laser Surg Med. 2006; 38: 150-154.

The most resent techniques provide the use of High Intensity Focused ultrasound (HIFU). FIG. 2 provides indicative regions where the various non-invasive (thermal) techniques aim to deposit their energies. Lasers cover the more superstitional skin layers, while RF penetrates into dermis parts of the skin.

### 3. Tissue Response to Ultrasound

Absorption of acoustic energy produces heat and may also induce the generation of cavitation. Cavitation are micro bubbles within the tissue which implode causing additional heating. The objective of applying HIFU in a skin rejuvenation context, is to elevate the local temperature to approximately 65 degrees C., thus inducing collagen contraction, Am J Sports Med. 1997; 25(1): 107-112. By targeting discrete volumes within dermal and other tissues by HIFU, this causes local thermal coagulation points and sparing adjacent tissues. In addition to coagulation, the application of targeted ultrasound energy (heat), causes collagen fibers in the subcutaneous tissues to denature and contract. The mechanism behind this is the breaking of intra molecular hydrogen bonds, causing the chains of collagen to fold. The biophysical consequence will be shorter, thicker and more stable collagen, in addition to the regeneration of (new) viscoelastic collagen (neocollagenesis) forms, resulting in lifting and tightening of skin laxity, Aesthetic Plast Surg. 2008; 32: 111-115, ClinCosmInv Derm. 2015; 8: 47-52.

Any aspect of collagen contraction, collagen intra molecular rearrangements and/or neocollagenesis are covered or included by the concept of rejuvenation.

### 4. State of the Art Related to HIFU and Rejuvenation

Ulthera Inc has developed a hand-held device or wand with fixed focal depth and interchangeable transducers, outlined in e.g. US 201503211026 and AU 2015238921, and spacers. The spacers are simple layers of fabric to vary the focus depth of the transducer(s) within the skin, by physically altering the distance from the transducer to the alleged point for the deposit of the energy. The procedure is to manually apply a Thermal Injury Zone (TIZ) along a defined straight 25 mm line, 0.5-5 mm apart, with 3 mm between each line. Short pulse durations are applied (25-50 ms), with relatively low energy (0.4-1.2 J/mm²). The transducers have fixed frequencies at 7.5 MHz [3 and 4.5 mm focal depth*)] and 4.4 MHz [4.5 mm focal depth*)]. Later, a 10 MHz transducer with focal depth of 1.5 mm was introduced to provide more superficial dermal neocollagenesis. The Ultrasound was locally applied and operated by the therapist by a push button located on the wand. *) With reference to paragraph 4, focal depths are not consistent with skin thickness data as provided in paragraph 1, or as skin data as displayed on Ulthera displays. A partial explanation can be that the transducer base line is located within the hand-held probe.

FIG. 3 shows the possible locations of manually located and operated treatment lines to the face. The trigeminal nerve and its branches are notified due to negative consequences due to possible maltreatments.

In AesthetSurg J. 2014 September; 34(7):1099-1110 the authors reported tightening and lifting of cheek tissue, improvement in jawline definition, and reduction in submental skin laxity in patients treated with HIFU.

A total of 103 adults were enrolled in a nonrandomized clinical trial. Three-dimensional photographs obtained at baseline and 3 months post treatment, were assessed qualitatively by 3 blinded reviewers and quantitatively with AutoCAD software.

93 patients were evaluated. Blinded reviewers observed improvement in skin laxity in 58.1% of the patients. During quantitative assessments, overall improvement in skin laxity was noted in 63.6% of evaluated patients. At day 90, 65.6% of patients perceived improvement in the skin laxity of the lower half of their face/neck. At day 90, improvements were reported by two-thirds of patients and by nearly 60% of blinded reviewers. Outcomes were better in patients with BMI≤30 kg/m².

In US 2010/0036292 a HIFU system and method is applied by transmitting one or more test signals into patient tissue and receives signals created in response to the test signals. The signals are analyzed to determine a response curve of how characteristic of the signals vies with the one or more test signals. The response curve of the detected signals is used to select a treatment parameter.

### 5. Technological Challenges to Optimal HIFU Rejuvenation

By assuming a universal skin thickness as the Ulthera equipment and procedures imply, and applying a standard transducer with a fixed focus point, which is manually moved and operated, the application of a predetermined (universal) energy deposit level will at best provide sub optimal treatment conditions and results. The risks for destroying subcutaneous fat layers are severe, thus inducing more wrinkles.

The objectives of the herein novel and inventive skin rejuvenance systems, are e.g. provide the fully automated system;
Diagnostic or measurements of the skin thickness in question,
2D or 3D digital mapping of the targeted skin volumes,
Variable focal depth, and/or focal range (length), and the optimal application and location (focal depth) of heat deposit points or volumes (focal range) within the skin or tissue(s),
(The detection of cavitation),
Measurements of variations in elasticity and/or tissue parameters to implicitly measure temperature changes and/or to optimized adequate energy deposits.
Provide optimal treatment without hazards to subcutaneous fat layers.

### 6.1 Supporting Technologies to Fulfil the Objectives of the Invention

Temperature affects the skin and tissue parameters.

Multiple ultrasound elastography techniques have been developed, and which rely on ARF in monitoring HIFU therapy. ARF is dependent on tissue attenuation and sound speed, both of which are known to change with temperature. Furthermore, the viscoelastic properties of tissues are also temperature dependent, which affects the displacements induced by ARF.

E. g. Phys. Med. Biol. 61 (2016) 7427-7447 and IEEE Trans Ultrasound FerroelectricFreq Control. 2013 April; 60(4): 685-701 discuss several techniques to monitor acoustically induced (elasticity) properties and/or parameters of tissues, enabling to e.g. calculate temperature, among them;

### Quasi-Static Methods

Acoustic Streaming in Diagnostic Ultrasound
Sonothermometry

### Transient Methods

Acoustic Radiation Force Impulse (ARFI) imaging
Shear Wave Elasticity imaging (SWEI)
Supersonic Shear Imaging (SSI)
Shear Wave Spectroscopy (SWS)
Spatially Modulated Ultrasound Radiation Force (SMURF)

### Harmonic Methods

Vibro-acoustography
Harmonic Motion Imaging (HMI)
Shear Wave Dispersion Ultrasound Vibrometry (SDUV)
Crawling Wave Spectroscopy (CWS)

Several supporting ultrasound techniques and technologies have been developed by the Applicant and/or Inventors, e.g.;

CN104125801 presents methods and instrumentation for measurement or imaging of a region of an object with waves of a general nature, for example electromagnetic (EM) and elastic (EL) waves, where the material parameters for wave propagation and scattering in the object depend on the wave field strength. The methods are based on transmission of dual band pulse complexes composed of a low frequency (LF) pulse and a high frequency (HF) pulse, where the LF pulse is used to nonlinearly manipulate the object parameters observed by the co-propagating HF pulse.

In EP2613171 methods and instruments for suppression of multiple scattering noise and extraction of nonlinear scattering components with measurement or imaging of a region of an object with elastic waves are developed. At least two elastic wave pulse complexes are transmitted towards said region where pulse complexes are composed of a high frequency (HF) and a low frequency (LF) pulse with the same or overlapping beam directions and where the HF pulse is so close to the LF pulse that it observes the modification of the object by the LF pulse at least for a part of the image depth. The methods are applicable to elastic waves where the material elasticity is nonlinear in relation to the material deformation. In CH101965232 acoustic probes that transmits/receives acoustic pulses with frequencies both in a high frequency (HF), and a selectable amount of lower frequency (LF1, LF2, . . . , LFn, . . . ) bands, where the radiation surfaces of at least two of the multiple frequency bands have a common region. The arrays and elements can be of a general type, for example annular arrays, phased or switched arrays, linear arrays with division in both azimuth and elevation direction.

US 20130096595 describes a system and methods to provided thrombi treatments in which hyperthermia is induced in an initial phase and cavitation and/or drug release are induced in a subsequent phase in a region of interest in a human or animal body. The system includes an energy transmitter having a variable intensity and/or a variable frequency; and a control unit arranged to control the energy transmitter to operate in at least two different modes. The initial hyperthermia treatment enhances the effect of subsequent treatments.

### 6.2 Ultrasound and Drugs—Treatment of Other Diseases

Sonoluminescence can occur when a sound wave of sufficient intensity induces a gaseous cavity within a liquid, and suffers a sudden collapse. The subsequent light flashes from the collapsing bubbles are extremely short, between 35 and a few hundred picoseconds long, with peak intensities of the order of 1-10 μW. Spectral measurements have given bubble temperatures in the range 2300 K to 5100 K, https://en.wikipedia.org/wiki/Sonoluminescence.

Sonodynamic therapy (SDT) is an emerging approach that involves a combination of low-intensity ultrasound and specialized chemical agents known as sonosensitizers. Ultrasound can penetrate deeply into tissues and can be focused into a small region of a tumor, to activate a sonosensitizer which offers the possibility of non-invasively eradicating of solid tumors, Cancer Biol Med. 2016 September; 13(3): 325-338. Examples of SDT areporphyrin-based sonosensitizers, xanthene-based sonosensitizers, non-steroidal anti-inflammatory drug-based sonosensitizers, and others like; curcumin, indocyanine green (ICG), acridine orange, hypocrellin B, 5-ALA, and/or (PDT) methylaminolevulinate. Diseases to be treated are, but are not limited to, acne, thrombi and cancers.

Multilevel rejuvenation of the face, neck, and décolletage can be obtained by enhancing volume restoration, neocollagenesis, and tissue contraction with combined efficacy of poly-L-lactic acid (PLLA) and HIFU. Concurrent treatment with PLLA and HIFU have been reported to be performed efficiently and safely, PlastReconstr Surg. 2015 November; 136(5 Suppl):180S-187S.

### SUMMARY OF THE INVENTION

An object of the present invention is to provide a system for treating wrinkles and rejuvenating skin that overcomes the problems of the prior art.

In a first aspect of the invention there is provided a system for treating wrinkles and rejuvenating skin on a human body, the system comprising a diagnostic component and a therapeutic component, an ultrasound probe, wherein the diagnostic component and the therapeutic component are connected to the ultrasound probe for diagnosis and therapy, a processor, and a memory. The processor running a program stored in the memory causing the system to perform the steps of obtaining an image of a depth of the skin in a region of interest on the skin using the diagnostic component, and, at each target point of a plurality of target points in the region of interest, determining how many ultrasound therapy foci to apply and the depths of each of the ultrasound therapy foci based on the image, and applying the ultrasound therapy foci at each of the depths using the therapeutic component.

According to another aspect of the present invention, the ultrasound probe includes i) a single ultrasound transducer or transducer array that is used by both the diagnostic component and the therapeutic component, ii) separate transducers or transducer arrays used respectively for the diagnostic component and the therapeutic component, and angled so that diagnostic beams and therapeutic beams overlap in the skin, or iii) separate transducers or transducer arrays used respectively for the diagnostic component and the therapeutic component, the separate transducers or transducer arrays being mounted in an acoustic stack or an annular structure so that a diagnostic beam axis and a therapeutic beam axis overlap.

According to another aspect of the present invention, the ultrasound probe transmits diagnostic ultrasound at a diagnostic frequency (DF) and therapeutic ultrasound at a therapeutic frequency (TF), wherein DF≥1.5·TF.

According to another aspect of the present invention, the processor running the program causes the system to further perform the steps of determining whether a skin thickness at the each target point is greater than a predetermined minimum thickness based on the image, if the skin thickness at the each target point is greater than a predetermined defined minimum thickness, then performing the steps of determining and applying at the each target point, and if the skin thickness at the each target point is not greater than a predetermined minimum thickness, then not performing the steps of determining and applying at the each target point.

According to another aspect of the present invention, the processor running the program causes the system to further perform, during the step of applying, measuring variations in a tissue parameter at the location of the each of the ultrasound therapy foci using the diagnostic component, and ceasing the step of applying of the each of the ultrasound therapy foci when the variations in tissue parameter meet a predetermined value.

According to another aspect of the present invention, the image obtained is a 3D image of the depth in the region of interest.

According to another aspect of the present invention, the image obtained is a 2D image of the depth obtained along a line in the region of interest. The processor running the program further causes the system to move the transducer in a direction orthogonal to the line and repeat the steps of obtaining along additional lines to obtain a plurality of 2D images that are combinable to form a 3D image of the region of interest. The steps of determining and applying are performed for each of the additional lines. More specifically, the steps of determining and applying are performed for each of the 2D images before a successive one of the 2D images is obtained.

The transducer may move within the probe in the direction orthogonal to the line to obtain the plurality of 2D images. Alternatively, the system can include a robotic arm on which the probe is mounted, the robotic arm capable of positioning and orienting the probe, wherein the robotic arm moves the transducer in the direction orthogonal to the line to obtain the 3D image.

According to another aspect of the present invention, a holding fixture is provided on which the probe is mounted. The holding fixture is capable of maintaining the probe at a fixed position for at least one of obtaining the image and applying the ultrasound therapy foci in a locked position and manually adjustable to change an orientation or position of the probe in an unlocked position.

According to another aspect of the present invention, a robotic arm is provided on which the probe is mounted, the robotic arm capable of positioning and orienting the probe, wherein the robotic arm moves the transducer for at least one of obtaining the image and applying the ultrasound therapy foci.

According to another aspect of the present invention, the diagnostic component is used during the step of applying the ultrasound therapy foci to correct for body movements.

The variations in the tissue parameter may include changes in an elastic stiffness of the tissue or changes in an optical property of the tissue.

According to another aspect of the present invention, the elastic stiffness is measured using an acoustic radiation force (ARF) mode of the diagnostic component to displace the skin.

According to yet another aspect of the present invention, the probe includes an acoustic standoff providing acoustic contact to a region of a multi-curved skin surface with low absorption so that a high ultrasound intensity is obtained in a subcutaneous focus region.

Further, the invention describes systems and their use of such systems for the treatment of wrinkles, acne, lipo sculpturing or causing the rejuvenation of the skin, comprising at least one diagnostic unit, at least one energy source, at least one processing unit (PU), wherein the system is characterized by:
mapping of tissue area and depths and the 3D mapping of tissues constituting regions of interest,
endogenously generated variable focal depths of therapeutic or diagnostic ultrasound probes,
endogenously generated measurements of variations in tissue (elasticity) parameters of tissues between the surface of the skin and throughout the region of interest,
endogenously generated application and the location of energy or heat deposit points or volumes within the region of interest,
cease the energy transmissions and/or heat deposits according to variations in elasticity parameters within or outside the regions of interest.

A patient is typically placed in a relaxed and fixed position in a chair or on a bench. The face and neck are supported, enabling the head and upper torso to stay in a fixed position for a defined duration of time. As an example, FIG. 4 describes a chair with an adjacent fixture which can support diagnostic and therapeutic units. The fixture and/or the diagnostic and/or therapeutic unit(s) can be fitted with positions sensor(s).

Region(s) of interest (ROI) is/are defined on the skin to be treated. The ROI(s) can be drawn by a digital pen, accompanied by one or several reference points. Preferably, the digital pen leaves a visual marking on the skin.

A computer calculates the surface x, y, z contour of the defined ROI. A diagnostic device is applied. The diagnostic device can be represented by combinations of analog or digital diagnostic imaging devices like X-ray, Computer Tomography, Magnetic Resonance Imaging, Positron Emission Tomography, ultrasound imaging and the like. Stereometric coordinates to one or several of the various skin layers, from the epidermis to muscle tissues or beyond, including the SMAS layer, are recorded with the use of the diagnostic device, and analyzed and mapped by a Processing Unit (PU), and subsequent skin volumes x, y, z contour(s) are established and labelled ROI*. The stereometric coordinates of the multiple ROI*(n), n=1, 2, 3 . . . are established by a PU with encompassing algorithms and software. FIG. 5 indicates the mapping of a surface contour. In combination with a diagnostic unit, a PU calculates stereometric coordinates of a skin volume.

In embodiments the diagnostic unit(s) and the energy (therapeutic) unit(s) are combined into one device mounted on a fixture or holder. In preferred embodiments the diagnostic and therapeutic units are represented by at least one combined ultrasound array. With reference to Chapter 1 of this document, typical foci distance from outer surface of a probe or dome/surface and the skin (epidermis) would be between 0.25 mm to approximately 1 mm [and applying one or two heat deposit zones in mid to lower dermis region(s)]. Assuming a 1 mm thickness of subcutaneous fat, a possible third heat deposit or thermal injury (or coagulation) zone would be approximately 2.2 mm from the skin surface, assuming a 1 mm skin (epidermis+dermis) thickness and a SMAS thickness of approximately 0.4 mm.

The invention provides a sub system or energy transmitter(s) to deposit energy and/or inducing hyperthermia within defined regions of the skin or tissue, comprising an energy transmitter having a fixed or variable intensity and/or variable frequencies; and a control unit arranged to control the energy transmitter. The control unit can be a PU.

It will be noted that several different heat sources could be used within this capacity of the invention. In some preferred embodiments, the energy transmitter comprises an electromagnetic energy transmitter. By applying this energy source, it can in some instances be desirable to use frequencies up to terahertz levels, preferably the electromagnetic energy transmitter is arranged to operate in frequencies between 100 MHz and 10 THz.

Preferably, the energy transmitter comprises (an) ultrasound transmitter(s). The ultrasound transmitter can be combinations of single transducers and an array of transducers. Single transducers may be focused by shaping the transducer. Arrays of transducers elements allow beam forming and focusing techniques to be used, e.g. for electronically steered the targeting of a defined ROI*. Preferably, the energy transmitter comprises a HIFU transmitter, more preferably with electronically steered focus depth and direction.

In preferred embodiments, the ultrasound transmitter is arranged to operate with a center frequency in the range of 0.3 to 100 MHz. Various frequencies can be used for different purposes.

In alternative embodiments, dual band ultrasound transducers can be used. Such transducers can be driven in either of two or more different frequency bands and can provide a greater separation between the frequencies used in the two modes of operation.

In other embodiments where ultrasound is used, the ultrasound unit can be used to monitor temperature, either directly or indirectly (calculated based on changers in physical or tissue parameters).

The energy transmitting unit can be placed on a fixture which can be manually and/or automatically controlled with the use of electronic, hydraulic and/or pneumatic means. In embodiments a robotically controlled arm with an energy transmitting device are controlled and guided, where data are processed by a PU with algorithms and subsequent software, to the desired locations where energy is/are to be deposited into the entire (multiple) ROI*s. The control unit, PU with algorithms and software, can deposit energy according to predetermined treatment programs or the actual treatment procedure, layout or design is manually or ad hoc defined for the treatment of the patient in question.

In most preferred embodiments, a combined array for treatment and array for imaging are located on an electronically controlled robotic arm fitted with position sensor(s).

The phase array for treatment operates in the 0.02 MHz to 250 MHz range, preferably in the 5 MHz to 75 MHz range.

The phase array for imaging (diagnostic) operates in the 0.5 MHz to 3 GHz range, preferably in the 10 MHz to 100 MHz range.

An area (or several areas) of interest (ROI) is defined (mapped or drawn) on the patient (FIG. 5). The PU calculated a volume of interest (ROI*) based on input (thickness and structure of the skin or tissues in question) from the diagnostic or imaging unit, and from the mapping device and software, represented by an analog or digital placed device (pen), which is moved over the skin. The PU will map the volume of interest (ROI*) by defining a mathematical mesh or defining digitally finite numbers of points or coordinates covering the ROI*. The coordinates can be 0.01 mm, 0.1 mm, 0.5 mm or other distances apart in the x, y, z directions.

The transducer(s) [phase array] for treatment can, guided by the PU and algorithms, provide energy in a predetermined mode, at e.g. two locations within the dermis layer of the skin, at a z distance 1 mm apart, and at one location within the SMAS layer.

Each x-y location to be treated can be spaced (e.g.) 1 mm apart. When one line of treatment is automatically completed, the PU will space (e.g.) 1 mm to the next line of treatment. It is possible to manually define on an ad hoc basis the spacing between each treatment point, between each treatment line, the spacing or location between each point or volume to deposit energy (in x-z direction).

The PU will electronically move treatment from one line to the next until the whole region—ROI or total volume ROI* is treated.

The PU will by the use of quasi-static, transient, harmonic methods or others, apply energy until changes in acoustic elasticity properties are recorded to be in consistent with a temperature increase of approximately 65 degrees C., or any other predetermined elasticity property value is achieved.

The system will automatically treat the entire ROI*.

The ROI* can be a cancer tumor or a thrombus located anywhere within a human body.

The ROI* can also represent the surface area of the skin to treat e.g. acne or superficially located cancers.

Energy, ultrasound based, light, RF, can be combined with drugs; sonosensitizers or others, to treat wrinkles, to cause rejuvenation.

For treatment of skin wrinkles it is important that the depth range of high heat generation in the skin tissues is short (<around 500 μm) so that one obtains heat deposition in selected skin tissue layers only. This can be achieved with an array, retracted ˜10 mm from the skin in a fluid bath connecting to the skin through a dome. The array retraction from the skin allows for a larger aperture of the array that allows for higher power transmission with a low F number, FN=F/D≈1 where F is the depth of the azimuth focus, and D is the azimuth transmit aperture width.

Based on historic developments with two-dimensional (2D) scanning of the beam, the scan direction is defined as the azimuth direction and the direction normal to this as the elevation direction. For an array with equal aperture dimensions in the azimuth and elevation directions, which for example can be obtained by one of an annular array, a matrix array, a 1.25 D-1.75 D array, or combinations of the above, known to anyone skilled in the art, the axial extension of the heat beam transmit focus is then for linear elasticity approximated as

Δr_{F}≈8λ(FN)²≈280 μm @50 MHz (1)

where λ=c/f≈30 μm is the wavelength, with a propagation velocity c≈1560 μm/μs and a frequency f=50 MHz. With such an aperture one gets a very sharp depth resolution, for example as shown in FIG. 7 which shows a simulation of the heat deposition in the tissue at a center frequency of 50 MHz. (701) shows the front of the dome at 7 mm from the array, and (702), (703) and (704) show localized heat deposition regions in W/mm³ with foci set at 7.25 mm, 7.75 mm, and 9.2 mm depth, respectively. The x-direction is the azimuth direction and the y-direction is the elevation direction, and we see that the dimensions of the heat deposition regions are the same in both directions due to the symmetry of the aperture in both azimuth and elevation directions of the annular array.

For the simulations one has used 0.036 dB/mmMHz for the dome (1 mm thick) and 0.052 dB/mmMHz, and an F-number FN=1.0012. The peak heat deposition is 309 W/mm³ for the 1st region, 178 W/mm³ for the 2^{nd} region and 45 W/mm³ for the 3^{rd} region. The drop in heat deposition is due to the absorption in the tissue, and can be compensated for by increasing the transmit power with the depth of focus. Note that due to the sharp focusing of the beam, the main heat deposition density is in the focal region, with very low heat deposition density in front of the focal region, for example as strongly shown for the deepest focus (704). Note that the short axial extension of the focus is according to Eq. (1) obtained with a low F-number (˜1) and the high frequency (50 MHz) giving the short wavelength λ≈30 μm.

With a 1 D linear array comprised of a single line of elements, the elevation focus Fe and aperture width De are fixed, giving a fixed elevation F-number, FNe=Fe/De. To cover a depth-range of foci then requires a reduced FNe for example FNe≈2 as used in the simulation shown in FIG. 8. Analogous to FIG. 7, this Figure show as (801) the front of the dome at 7 mm depth, and (802), (803), (804) shows the heat deposition intensities with azimuth focus set at 7.25 mm, 7.75 mm, and 9.2 mm depth, respectively, and an azimuth F-number set at FNa=Fa/Da≈1 for each depth, where Fa and Da are the azimuth focus and aperture width, respectively.

With the sharp focusing of the treatment beam the pressure amplitude increases rapidly as the pressure wave enters the focal region. This high pressure introduces a large nonlinear distortion of the focal pulse in the focal region, which introduces a large degree of higher harmonic bands in the pulse, increasing the total power absorption, and hence heat generation from the pulse.

One notes that with a fixed elevation focus and aperture in FIG. 8 the range definition of the heat deposition regions is lower than with the lower range (169 W/mm³ for the 1st region, 106 W/mm³ for the 2^{nd} region and 24 W/mm³ for the 3rd region) steered elevation focus and aperture width with FNe≈1 as shown in FIG. 7. However, we still have an interesting range resolution of the heat deposition region, which makes the solution with a 1 D linear array useful. The range resolution can be adjusted by at least one of the F-numbers and the frequency (wavelength). Due to heating of the ultrasound transmit array, the transmit power intensity on the array surface is often limited. By increasing distance of the array from the dome, one can then increase the aperture width with the same F-number, for example to increase the total heat deposition with limited transmit power intensity on the array surface.

For imaging of the tissue structures prior to treatment, for example to determine focal depth positions, number of heat deposit regions, and range extensions of heat deposit regions, one can use high frequency ultrasound measurements or imaging of the tissue structures. In this case the image range resolution is determined by the transmitted pulse length, and the resolution along the beam axis (range resolution) is appr 1.3 wavelengths, λ. With λ=c/f₀ where c=1540 μm/μs we get a range resolution of Δr≈1.3λ≈2000/f₀ μm, where f₀ is the center ultrasound frequency in MHz. For f₀=30, 40, and 50 MHz we get Δr=67, 50, and 40 μm. This resolution is so sharp that ultrasound pulse echo measurements of the tissue structures can be used to determine the above heat deposition parameters.

Multiple reflections in the dome can increase the effective transmitted pulse length, and hence the resolution in the measurements of the tissue layers. These multiple reflections in the dome can according to the invention be reduced with acoustic matching layers on at least one side of the dome.

Heating of the tissue produces changes in ultrasound tissue properties such as in Suomi. V. et al: “The effect of temperature dependent tissue parameters on radiation force induced displacements.” Phys. Med. Biol. 61 (2016) 7427

i) In Suomi. V., et al it is shown an increase in sound speed in liver of ˜1% with a temperature increase from 37° C. to ˜65° C. This will move the scatterer delay past the region of coagulation of the order

$$Δ t = \frac{2 r}{{c}_{2}} - \frac{2 r}{{c}_{1}} = \frac{2 r}{{c}_{1}} ( \frac{1}{1.01} - 1 ) \approx - 3.8 ns \qquad ( 2 )$$

for r=300 μm and c1=1560 μm/μs. With a center frequency of 50 MHz the RF oscillation period is T=20 ns, which gives λt/T≈0.2 which is well in the observable range with modern phase detection techniques. Measurement of signal phase changes from the region beyond treatment is hence useful, albeit details of the variation of sound speed with temperature in the various skin regions is required.
ii) In Suomi. V. et al it is shown that ultrasound absorption in liver increases with temperature ˜0.5 dB/cmMHz from ˜45-65 degree Celsius. For 50 MHz the absorption over 0.3 mm increases ˜0.75 dB, which gives a reduction of signal amplitude passed the treated region of −0.75 dB˜0.92. This increase in attenuation is low, and it is questionable if it is sufficient to monitor the temperature increase and required tissue changes (coagulation).
iii) Scattering of ultrasound from the treatment region also changes with changes in tissue temperature and tissue composition and can be used for estimation of changes in tissue structure.
iv) Increase in temperature and tissue coagulation also increases the tissue shear stiffness. This increase in shear in shear stiffness can be measured from phase change in the received signal produced by tissue movement from ultrasound radiation force.

The accuracy of tissue temperature measurement and assessment of coagulation change in the tissue structure can be increased by combining two or more of these measurements.

For all energy deposits (foci depths and or ranges), as indicated by but not limited by, (702), (703), (704), (802), (803), (804) in FIGS. 7 and 8, the frequencies, sequencing, exposure times, energy levels, induced or implied temperatures, can vary.

Multiple photon scattering in human skin limits the optical penetration depth into the tissue. As a consequence, optical observation of tissue temperature and tissue changes due to ultrasound heating can only be done close to the tissue surface. However, in our application, the ultrasound treated tissue is close to or at the surface of the body or organs, and optical techniques are thus useful for observing changes in both temperature and in tissue composition.

The optical techniques suitable for such monitoring can be divided into three groups:

i) Passive observation of emitted infrared radiation intensity, typically in the wavelength ranges 3-5 μm or 7-10 μm [reference 1, 2, 9]. The tissue will emit radiation as a black or grey body and changes in temperature will alter the emission profile. Temperature changes in the tissue in the ultrasound heated region can thus be detected as changes in the emitted infrared radiation. The radiation intensity can be measured with small, spatially resolved infrared sensor arrays. One can buy off-the-shelf infrared cameras that are useful for such purpose, potentially with a modification of the lens focusing system, an example of such a compact device intended for attachment to mobile phones can be found in [3].
ii) Local tissue changes caused by heating alters the scattering/absorption in the modified tissue [4, 5, 6, 7, 8, 14]. One can hence indirectly observe local changes in tissue temperature by monitoring changes in the optical properties of the tissue constituents. This can be achieved by measuring the reflected light intensity from the tissue using a light source of a given wavelength to illuminate the tissue, typically in the visible wavelength range (400-800 nm). Such measurements can be achieved by several techniques such as optoacoustic imaging [10], optical coherence tomography [8] or by measuring changes in the bulk optical properties of the tissue using miniature spectrometers or by using a generic optical sensor equipped with and appropriate optical filter (typically a bandpass filter).
iii) Ultrasound heating will be accompanied by changes in the emitted fluorescence of certain molecules due to structural changes in the molecules themselves [11, 12, 13]. Typical excitation wavelengths for detecting such changes are in the ultra violet and blue wavelength ranges, with fluorescence emission in the visible wavelength range. This fluorescence can be detected by several means such as fluorescence spectroscopy using an optical detector equipped with a filter suitable for the fluorophore in question.
1. DOI: 10.1109/MEMB.2002.1175137
2. Integr Cancer Ther. 2009 March; 8(1):9-16. doi: 10.1177/1534735408326171.
3. http://www.flir.com/flirone/ios-android/4.
4. Int J Hyperthermia. 2011; 27(4):320-43. doi: 10.3109/02656736.2010.534527.
5. DOI: 10.1109/IEMBS.1989.96158 ⋅ Source: IEEE Xplore
6. Ritz, J.-P., Roggan, A., Isbert, C., Müller, G., Buhr, H. J. and Germer, C.-T. (2001), Optical properties of native and coagulated porcine liver tissue between 400 and 2400 nm. Lasers Surg. Med., 29: 205-212. doi:10.1002/Ism.1134
7. DOI: 10.1109/10.508546
8. Biomed Opt Express. 2015 Feb. 1; 6(2): 500-513. Published online 2015 Jan. 12. doi: 10.1364/BOE.6.000500
9. Sensors 2014, 14, 12305-12348; doi:10.3390/s140712305
10. J. Phys. D: Appl. Phys. 38 (2005) 2633-2639 doi:10.1088/0022-3727/38/15/015
11. DOI: 10.1002/(SICI)1096-9101(1997)20:3<310::AID-LSM10>3.0.CO; 2-H
12. Lasers Surg Med. 2012 November; 44(9):712-8. doi: 10.1002/Ism.22080. Epub 2012 Oct. 4.
13. http://www.springer.com/gp/book/9789048188314?wt_mc=GoogleBooks. GoogleBooks.3.EN&token=gbgen
14. Lasers Surg Med. 2004; 34(5):414-9.OPEN

### BRIEF DESCRIPTION OF THE DRAWINGS

In the drawings,

FIG. 1 is a cross-sectional view of human skin;

FIG. 2 is a cross-sectional view of human skin showing a prior art ultrasound probe;

FIG. 3 is perspective view of a human face and a side sectional view showing line placement and location of Trigeminal nerve;

FIG. 4 shows a chair and robotic arm according to an embodiment of the present invention;

FIG. 5 is a perspective view of a computer simulation of a face mapping a Region of Interest according to an embodiment of the present invention;

FIG. 6a shows various transducer array shapes that can be used according to the present invention;

FIG. 6b is a diagrammatic view showing a transducer according to the present invention;

FIG. 6c shows schematic diagrams depicting various embodiments of the transducer according to the present invention;

FIG. 7 shows a simulation of the heat deposition in the tissue according to an embodiment of the present invention;

FIG. 8 shows a simulation of the heat deposition in the tissue according to another embodiment of the present invention;

FIG. 9 is a schematic block diagram of an embodiment of the system of the present invention; and

FIG. 10 is a schematic diagram showing the locations of therapy foci (energy deposits) according to an embodiment of the present invention.

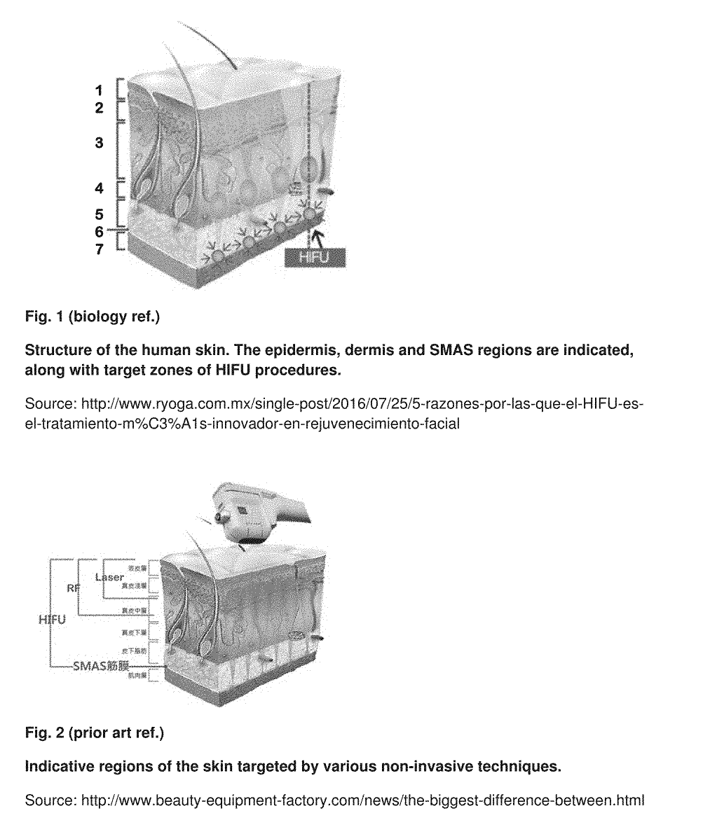

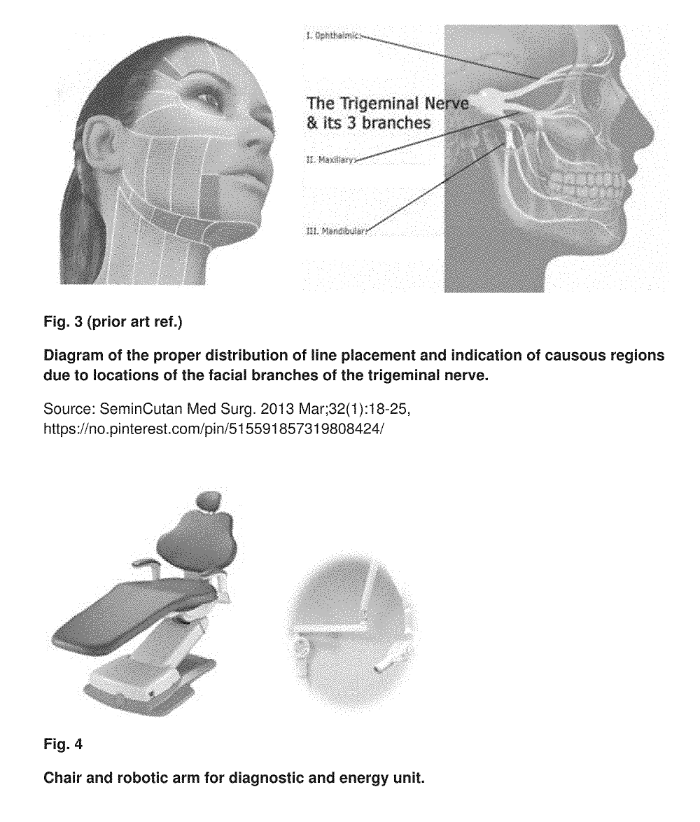

### DETAILED DESCRIPTION OF THE INVENTION

A patient is typically placed in a relaxed and fixed position in a chair or on a bench, related to (rejuvenation) treatment. The face and neck are supported, enabling the head and upper torso to stay in a fixed position for a defined duration of time. As an example, FIG. 4 describes a chair with an adjacent fixture or robotic arm which can support diagnostic and therapeutic units. The fixture or robotic arm and/or the diagnostic and/or therapeutic unit(s) can be fitted with positions sensor(s).

In other treatment modi, like the treatment of diseases like thrombi or cancers, the patient can favorably be placed in a bed or in other positions. (Approximate) real time treatment is a preferred modus operandi, but recording of and/or the analysis or definition(s) of ROI*s can be performed before the actual treatment or application or deposit of energy, with or without the application of drugs.

One or several regions of interest (ROI) is/are defined on the skin to be treated. The ROI can be drawn by a digital pen, accompanied by one or several reference points and/or other positioning devices. Preferably, the digital pen leaves a visual marking on the skin.

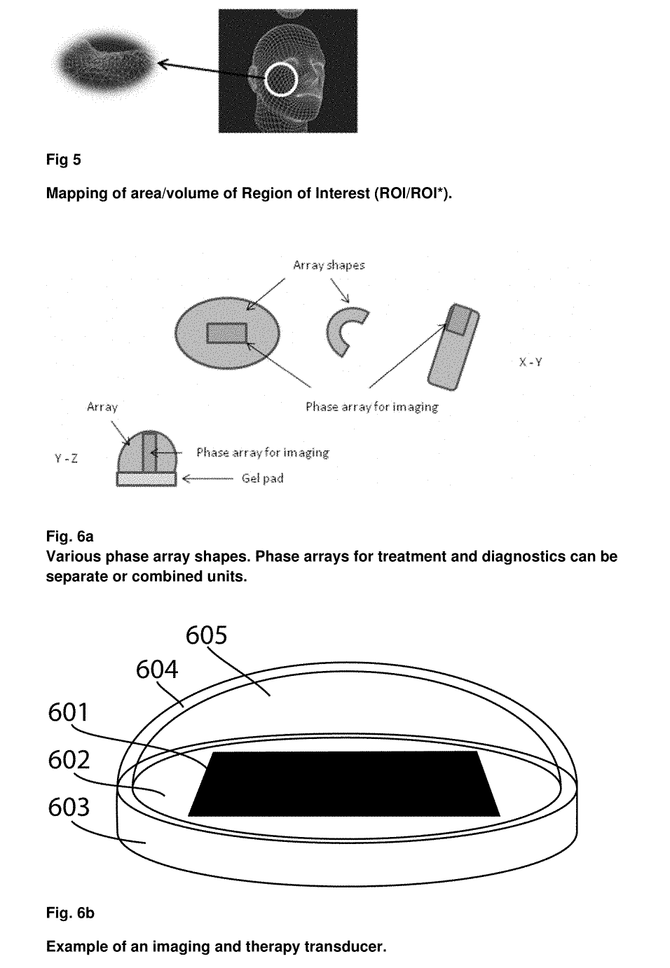

A computer (and/or PU) calculates the surface x, y, z contour of the defined ROI. A diagnostic device is utilized. The diagnostic device can be represented by combinations of digital or analogous diagnostic imaging devices like X-ray, Computer Tomography, Magnetic Resonance Imaging, Positron Emission Tomography, ultrasound imaging and the like. Stereometric coordinates to one or several of the various skin layers, from the epidermis to muscle tissues or beyond, including the SMAS layer, are recorded with the use of the diagnostic device, and analyzed and mapped by a PU, and subsequent skin volumes x, y, z contour(s) are established and labeled ROI*. The stereometric coordinates of the (multiple) ROI*(n), n=1, 2, 3 . . . are established by a PU with encompassing algorithms and software. FIG. 5 indicates the mapping of a surface contour. In combination with a diagnostic unit, a PU calculates stereometric coordinates of a skin volume.

In embodiments the diagnostic unit(s) and the energy unit(s) are combined into one device mounted on a fixture. In preferred embodiments the diagnostic and therapeutic units are represented by at least one ultrasound array.

The skin type and/or parameters of certain tissues or any treatment parameters can be exogenously stated. Exogenous is defined by factors which are caused, stated, produced or synthesized outside the organism or system under consideration. The ROI* can be located deep into a human or animal body representing thrombi, cysts, tumors, can be represented by fat tissues or the like. Interleaved imaging beams between therapy beams can be provided by diagnostic and/or therapeutic energy units to correct for potential body movements.

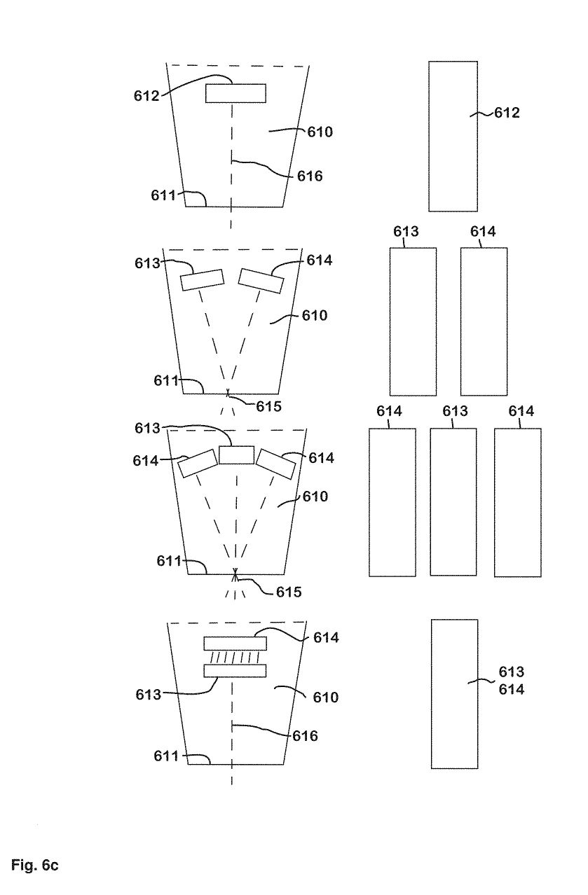

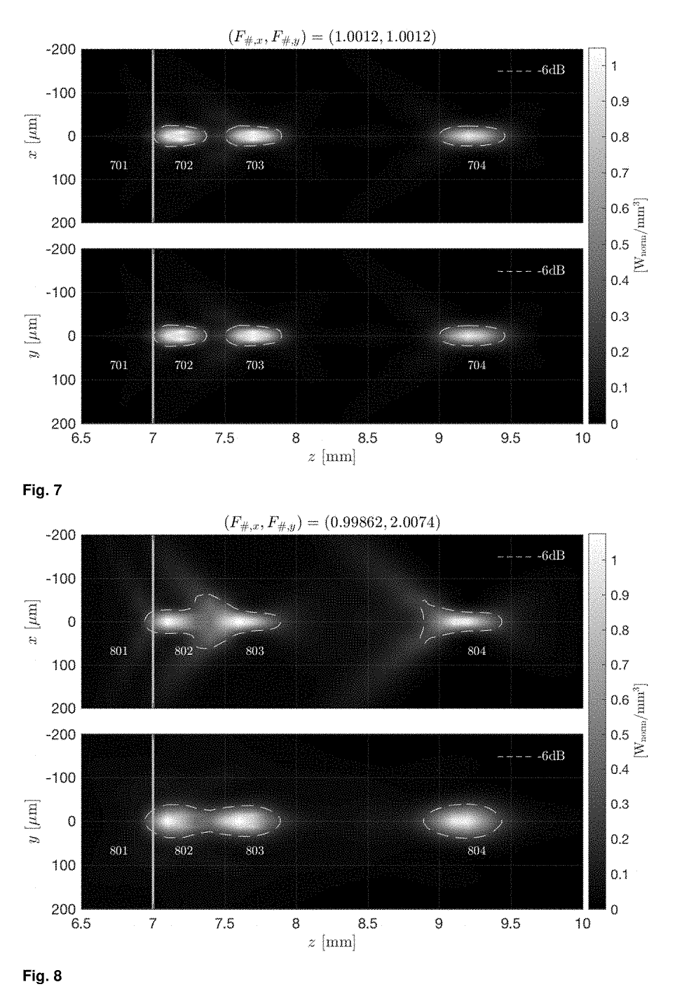

FIG. 6a outlines array shapes, dependent on where they are to be applied. Minor arc shaped arrays or transducers can be applied around the eyes or the mouth. Larger elliptically shaped arrays or transducers can be applied on the cheeks. A gel padding can be an integral part of the array, to provide added acoustic contact. An additional layer of gel can be applied between the gel padding and the skin.

The arrays and elements can be of a general type, for example annular arrays, phased or switched arrays, matrix arrays, linear arrays with division in both azimuth and elevation direction.

FIG. 6b outlines an example of a combined therapeutic and imaging transducer. The numbers stated on the figure represent, but are not limited to, the following;
601—Transducer aperture. Radiating surface. Therapeutic and Imaging. Imaging and therapy transducers are either further divided into two areas or stacked.
602—Transducer baffle.
603—Transducer backing. Non-active area.
604—Gel-membrane. In contact with the skin.
605—Gel-filled volume allowing direct contact to ROI.

FIG. 6c shows four other arrangements of the diagnostic and therapeutic transducers. The transducers are mounted in a fluid-filled compartment (610) with front dome material (611) that is in acoustic contact with the skin surface, according to known methods. The retraction of the transducer from the dome simplifies the design of high power transducers with low f-number focusing that gives a short (Re Eq. (1)) and narrow beam focus, both for diagnosis and therapy, according to known methods.

The Figure shows from top to bottom 4 attractive arrangements, where the left column figure sets show a cross section of the fluid filled compartments, the diagnostic, and the therapeutic arrays, while the right column figure sets show the diagnostic and therapeutic arrays seen from above. For illustration purpose linear arrays are shown, while it is clear to anyone skilled in the art that other types of transducers, such as single element transducers, annular arrays, curved linear arrays, phased arrays, 1.5 D arrays, 1.75 D arrays, and matrix arrays can be used, all known to anyone skilled in the art. For simplicity we shall refer to all forms of arrays as the transducer, which is a common term for a device that converts between acoustic and electric energies.

In the upper arrangement, the same transducer (612) is used both for diagnosis and therapy. Ultrasound transducers are band-limited, and this solution restricts the difference between the frequencies for diagnosis and therapy that can be used. There are also different restrictions in the optimization of the transducers for wide band imaging and high-power therapy, which in total produces a less than optimal performance with this solution.

The 2^{nd} upper arrangement shows a different transducer for diagnosis (613) and therapy (614) mounted side by side, and angled so that the beams overlap in the skin region (615). In the 3^{rd} arrangement from above, two therapeutic arrays (614) are mounted on each side of the diagnostic array (613). In the bottom arrangement, the diagnostic array (613) is stacked in front of the therapeutic array (614) with an acoustic isolation section between. Such a solutions described in U.S. Pat. No. 7,727,156 and U.S. Pat. No. 8,182,428 patents.

The advantage of the three lower arrangements is that the diagnostic and therapeutic arrays can be separately optimized both for frequency, aperture/focus, bandwidth and power, for optimal imaging and therapy. It is generally an advantage to use a higher diagnostic frequency (DF) than the therapeutic frequency (TF). If the same transducer is used for diagnosis and therapy as in the upper arrangement of FIG. 6c, the limited bandwidth allows DF/TF≈1.5. However, when separate diagnostic and therapeutic arrays are used as in the three lower arrangements of FIG. 6c, one has a larger freedom in selecting DF and TF up to say DF/TF≈5, where other practical concerns might force the ration down to DF/TF≈2. The weakness with the 2^{nd} and 3^{rd} upper arrangements in FIG. 6c, is that the angling of beams provides god beam overlap in a limited depth range. Two therapeutic arrays on each side of the diagnostic array in the 3^{rd} upper arrangement provides a narrow main-lobe of the therapeutic beam, with increased side-lobes. Solution of stacked diagnostic and therapeutic arrays in the lowest arrangement provides for optimizing both frequency, aperture/focus, bandwidth and power, with a common beam axis (616) for both imaging and therapy.

Separate diagnostic and therapeutic arrays can also be obtained by an annular structure, where for example the outer elements are used for the therapy and the inner elements are used for the diagnosis. This allows separate optimization of the therapeutic and diagnostic frequencies for different frequencies and apertures with the same beam axis. One should know that a ring structure gives an increase in side-lobe level for the outer array, albeit with a narrow main lobe. A layered structure of the therapeutic and diagnostic arrays as in the lower panel of FIG. 6c, can also be used with annular arrays, with the same advantages as for the linear arrays.

A fixture, holder or a robotic arm can be mounted on the (right) side of the device. Distance to the body can be measured by combinations of pressure gradients within the gel-volume (605) and ultrasound imaging (601).

The beam can be steered electronically from the aperture (601), also in combination with mechanical movement of the aperture. A square aperture (601) can be electronically controlled in three dimensions (elevation, azimuth and depth). Alternatively, also in combination with mechanical movements, the transducer (601) can be moved mechanically by (601) or (601), (602) and (603). The invention provides a sub system or energy transmitter(s) to deposit energy and/or inducing hyperthermia within defined regions of the skin, comprising an energy transmitter having a fixed or variable intensity and/or variable frequencies; and a control unit arranged to control the energy transmitter. The control unit can be a PU.

It will be noted that several different heat sources could be used within this capacity of the invention. In some embodiments, the energy transmitter comprises an electromagnetic energy transmitter. By applying this energy source, it can in some instances be desirable to use frequencies up to terahertz regions, preferably the electromagnetic energy transmitter is arranged to operate in frequencies between 100 MHz and 10 THz.

Preferably the energy transmitter comprises an ultrasound transmitter. The ultrasound transmitter may be combinations of single transducers, an array of transducers or a phase array of transducers. Single transducers may be focused by shaping the transducer. Arrays of transducers allow beam forming and focusing techniques to be used, e.g. for electronically steered the targeting of a defined ROI*. Preferably, the therapeutic component comprises a HIFU transmitter, more preferably with electronically steered focus depth and direction.

In preferred embodiments, the ultrasound transmitter is arranged to operate with a center frequency in the range of 0.3 to 100 MHz. Various frequencies can be used for different purposes.

In alternative embodiments, multiband ultrasound transducers can be used. Such transducers can be driven in either of two or more different frequency bands and can provide a greater separation between the frequencies used in the two modes of operation.

In other embodiments where ultrasound is used, the ultrasound unit can be used to monitor temperature, either directly or indirectly (calculated based on changers in physical parameters).

The energy transmitting unit can be placed on a fixture or a robotic arm which can be manually and/or automatically controlled with the use of electronic, hydraulic and/or pneumatic means. In embodiments a robotically controlled arm with an energy transmitting device are controlled and guided, where data are processed by a PU with algorithms and subsequent software, to the desired locations where energy is/are to be deposited into the entire (multiple) ROI*s. The control unit, PU with algorithms and software, can deposit energy according to predetermined treatment programs or the actual treatment procedure, layout or design is manually or ad hoc defined for the treatment of the patient in question.

In most preferred embodiments, a combined ultrasound probe for imaging and treatment is located on a mechanical and/or electronically controlled robotic arm. The array for treatment operates in the 0.02 MHz to 250 MHz range, preferably in the 5 MHz to 75 MHz range. To induce cavitation to liquefy or destroy fat tissue in lip sculpturing applications, frequencies in the 20 kHz to 2 MHz are preferred. The phase array for imaging (diagnostic) operates in the 0.5 MHz to 3 GHz range, preferably in the 10 MHz to 100 MHz range.

An area of interest (ROI) is defined (mapped or drawn) on the patient (FIG. 5). The PU calculated a volume of interest (ROI*) based on input (thickness and structure of the skin or tissues in question) from the diagnostic or imaging unit, and from the mapping device and software, represented by an analog or digital placed device (pen), which is moved over the skin. The CPU will map the volume of interest (ROI*) by defining a mathematical mesh or defining digitally finite numbers of points or coordinates covering the ROI*. The coordinates can be 0.01 mm, 0.1 mm, 0.5 mm or other distances apart in the x, y, z directions.

The transducer(s) [phase array] for treatment can, guided by the PU and algorithms, provide energy in a predetermined mode, at e.g. two locations within the dermis layer of the skin, at a z distance of e.g. 1 mm apart, and at one location within the SMAS layer.

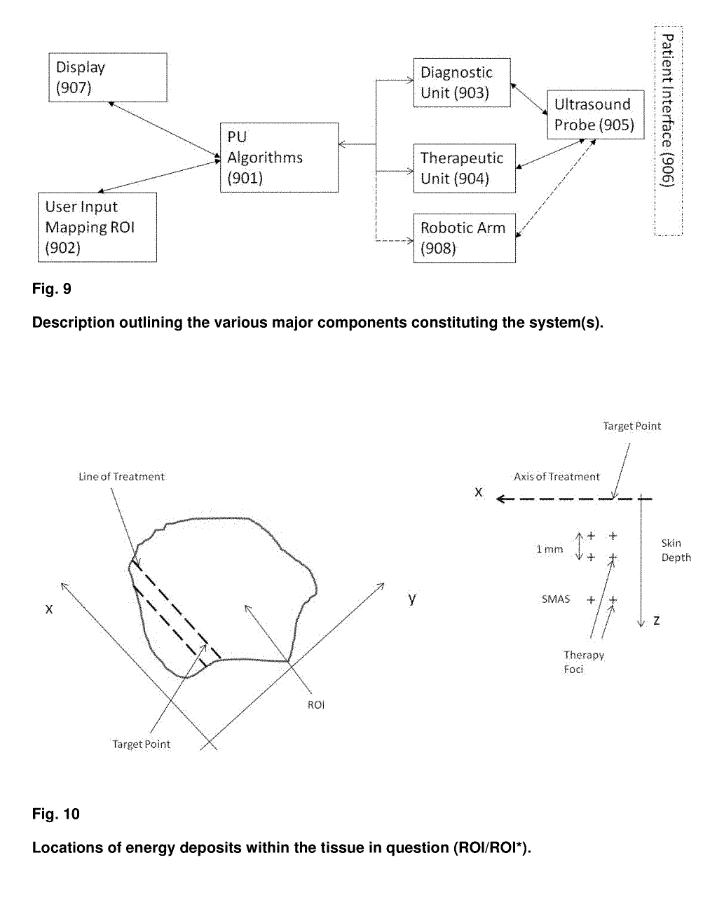

FIG. 10 indicates the lines of treatment in the x-y and x-z planers and target points.

Each x-y location to be treated can be spaced (e.g.) 1 mm apart. When one line of treatment is completed, the PU will space (e.g.) 1 mm to the next line of treatment. It is possible to manually define the spacing between each treatment or target point, between each treatment line, the spacing or location between each point or volume to deposit energy, which are labelled therapy foci in FIG. 10 (in the x-z direction).

Generally, the energy source and/or therapeutic transducer(s) with variable focal depth and/or focal range (length), can endogenously or exogenously deposit heat at variable deposit points (therapy foci) or volumes (focal range) within tissues. Focal depth is the distance to from the active surface of the transducer(s) or the energy unit(s) to the center of the heat point (therapy foci). Focal range is the beam axis length. A display unit can in real time display the treatment in combinations of x-y, x-z, y-z planes and other cross-sectional directions. The PU will electronically move treatment from one treatment line to the next until the whole region—ROI or total volume ROI* is treated.

The pattern of applying the energy deposits, can be squares, circles or any other geometric shape. The PU will by the use of quasi-static, transient, harmonic methods or others, apply energy until changes in elasticity properties are recorded to be in consistent with a temperature increase of approximately 65 degrees C., or lower or higher, if desired, or any other predetermined elasticity property value is achieved.

In an ablation mode of application, the temperature (increase) would normally be (up to) approx. 80 degrees C.

The system will automatically treat the entire ROI*.

Energy, ultrasound based, light, RF, can be combined with drugs; sonosensitizers or others, to treat wrinkles, to cause rejuvenation, and to treat diseases (acne, cysts, cancers, thrombi).

FIG. 9 shows a system according to the invention.

The system comprises a processing unit (PU), (901), that runs programs for steering the diagnostic and therapeutic processes. The PU takes user inputs from the user interface unit (902), for example from manual definition of therapeutic regions of interest (ROI), for example using a digital pen, or other user information such as specification of distance between target points, minimal thickness of dermis to be treated, etc. Based on this information, the PU steers the diagnostic (903) and the therapeutic (904) units that both connects to the ultrasound probe (905) for transmission and reception of diagnostic and therapeutic ultrasound signals from the probe into the patient skin (906). The ultrasound probe comprises at least one ultrasound transducer for transmission/reception of diagnostic and therapeutic ultrasound to the patient. The probe may in alternative embodiments also include an optical measurement system that senses optical tissue changes during treatment in a target point. The ultrasound transducer can be composed of a single element, and array of elements according to known methods, and as discussed in relation to FIG. 6c, and we shall in the following use the term transducer for all forms of conversion between electric and acoustic energies. In a preferred embodiment, separate transducers are used for diagnosis and therapy at different frequencies, as described in relation to FIG. 6c. In a preferred embodiment the transducer(s) are mounted in a fluid filled chamber of the probe retracted a distance from an acoustic layer (dome) that is in acoustic contact with the patient, according to known methods. The retraction of the transducer simplifies the design of high power transducers with low f-numbers that gives a short (Re Eq. (1)) and narrow beam focus, both for diagnosis and therapy, according to known methods.

In a simplest embodiment, the transducer is able to scan both the diagnostic and therapy foci along a line for 2D images of the skin with depth and perform therapy along an azimuth line (direction) of the skin surface. Mounting the transducer in a fluid filed chamber allows the use of single element or annular array transducers that require mechanical movement along the azimuth direction, according to known methods.

To form therapy across an area of the skin surface, the probe can for example be moved manually in an elevation direction normal to the azimuth line, or this movement can be done by a robotic arm as discussed below.

Placing the transducer in a fluid-filled chamber also allows lateral motion of the transducer in the elevation direction for scanning the diagnostic and therapeutic beams across a surface area of the skin for 3D imaging, where the probe contact to the skin is stationary. This is also the case for single element and annular array transducers that require mechanical movement of the array both for scanning in an azimuth direction along a line, and the elevation direction to scan the beam across a surface area.

The PU also connects to a display unit (907) to give inputs to the user, for example ultrasound images of the skin produced by the diagnostic unit, state parameters of the system and its operation, results of image analysis, etc.

Utilizing the user inputs, the PU at least

i) sets the system to acquire images with depth(z) of the skin, either in a 2D manner with scanning the diagnostic beam along an azimuth line (x) across the skin, or a 3D manner with additional scanning the diagnostic beam in an elevation direction (y) across a skin surface (x-y), and
ii) analyses the images to determine the dermis thickness in actual target points of treatment, and
iii) if the dermis thickness is above a set limit in a target point, the PU sets the system to transmit therapy beams in said target point, decides iiia) how many therapy foci and depths to be used in each target point and therapeutic power and maximum therapeutic time in each target point.

The PU also has the ability to

i) set the diagnostic unit to a mode to detect elastic changes in a treatment focus at intervals during the therapeutic transmission to set treatment focus, and cease the therapy transmission in said treatment focus when elastic changes reaches a limit, or
ii) utilize said optical system to observe optical changes in the treatment focus and cease the therapy transmission in said treatment focus when optical changes reach a limit.

For a more advanced embodiment, the ultrasound probe can be connected to a fixture (908), as exemplified in FIG. 4. The fixture can be locked and unlocked by the operator. In an unlocked state of the fixture, the probe can be moved by the operator to a desired position on the skin. Locking the fixture for this position of the probe, the fixture will keep the probe on the same position on the skin, as long as the patient does not move. To handle movement of the patient, motors can be added to the joint of the fixture so that it becomes a robotic arm that is able to follow movements of the patient. The robotic arm can also move the probe across the skin for treating a larger region of the skin, also with for example a probe that provides scanning of the diagnostic and therapy beams across a skin surface.

Endogenous effects and/or variables are caused by factors produced, established or synthesized within an organism or system.

In an additional embodiment a system and the use of such system, for the treatment of wrinkles and other diseases cause the removal of or provide the liquefying of fat tissues due to cavitation (lipo sculpturing) or causing the rejuvenation of the skin, within the human skin, comprising at least one diagnostic unit, at least one energy source, at least one central processing unit, wherein the system is characterized by:
in real time,
   measurements of tissue area and depths and the 2D or 3D mapping of tissues constituting regions of interest.

The algorithms and/or computer (PU) can further provide;
endogenously generated variable focal depths of therapeutic or diagnostic ultrasound probes,
(ultrasound transmitters for providing ultrasound therapy beams with steerable direction and focus depth across a selected therapy-region of said image-region),
endogenously generated measurements of variations in elasticity parameters of tissues between the surface of the skin and throughout the region of interest,
endogenously generated application and the location of energy or heat deposit points or volumes within the region(s) of interest,
cease the energy transmissions and/or heat deposits according to variations in elasticity parameters within or outside the regions of interest.

The system is further enabling to;
the energy source and/or therapeutic transducer(s), with variable focal depth and/or focal range (length), can endogenously or exogenously deposit heat at variable selectable deposit points or volumes (focal range) within tissues.
Interleaved imaging beams between therapeutic beams can be provided by diagnostic and/or therapeutic energy units to correct for potential body movements.

The system is further enabling to;
to directly or implicitly measure temperature changes in tissues and/or to provide adequate energy deposits data within the region of interest,
comprising one of the detection of cavitation or drug dosage supply and/or control within the region(s) of interest,

The system is further enabling to and/or comprising at least one of;
an ultrasound probe for transmitting and receiving imaging beams and transmitting therapy beams for a region of a skin surface,
ultrasound transmitters and receivers for providing a 2D or 3D ultrasound image of an image region of a skin surface,
   ultrasound transmitters for providing ultrasound therapy beams with steerable direction and focus depth across a selected therapy-region of said image-region,
steering said ultrasound transmitters and receivers to generate 3D ultrasound images of the image region of the skin surface and transmitting therapy beams across a selected therapy region,

The system is further enabling to and/or comprising at least one of;
analyzing the 3D images of said skin surface to determine depth of skin in said region,
select said therapy-region and therapy beam directions within said therapy-region,
select therapy beam focus for each therapy beam direction, and select transmit aperture of each said therapy beam directions,
select the transmit frequency of each said therapy beam directions, determine a transmit intensity of each therapy beams,
select a maximal transmit time for each therapy beam directions,
setting up said therapy beams in an acoustic radiation force (ARF) mode to push the skin surface while measuring the skin displacement with imaging beams to monitor displacement of the skin to monitor changes in elastic stiffness of the skin,
using changes in the measured elastic stiffness for each therapy beam direction as a measure to decide to stop the therapy beam transmission in said therapy beam direction,

The system is further enabling to and/or comprising at least one of;
where said ultrasound probe comprises a soft acoustic standoff with that provides acoustic contact to a region of a multi-curved skin-surface and with low absorption so that a high ultrasound intensity is obtain in the sub-cutaneous focus that provides a high degree of nonlinear distortion that reduces the axial extension of the region of high heating in the therapy beam focus,

The system is further enabling to and/or comprising at least one of;
where a set of therapy beams are directed with crossing directions and so that the foci of the individual beams overlaps to reduce the region of high intensity with high distortion of the ultrasound oscillation to reduce the region of high intensity acoustic heating.

System and/or method for cosmetic treatment of a region of skin (of a human) by use of ultrasound, representing combinations of;

Thickness measurements of various tissue layers, energy deposits and the application of ultrasound frequencies above 5 MHz.
E1. A method for cosmetic treatment of a region of skin (of a human) by use of ultrasound, the method comprises:
   using ultrasound therapy beams radiated onto a selected therapy region of the skin, and
estimating for each therapy beam direction the depth of skin tissue structures based on ultrasound measurement of skin tissue structures in selected 1st beam positions, an
from the estimate of the skin structures, determining at least one set of transmit parameters for at least one therapy beam, where said transmit parameters comprises at least one of i) a transmit focus, ii) a transmit frequency, iii) a transmit pulse amplitude, iv) a transmit pulse length, v) a transmit pulse repetition frequency, vi) a treatment beam transmit duration, and
setting up said transmit parameters for said at least one therapy beam and scanning therapy beams across the region of the skin to be treated.
E2. A method according to E1, combining said measuring of the skin thickness and skin composition with apriori information about the individual and the skin for determining at least one set of parameters for at least one therapy beam.
E3. A method according to E1, measuring beam parameters for each transmit beam position, or a group of transmit beam positions.
E4. A method according to E1, at least two transmit foci of the therapy beams
E5. A method according to E1, scanning 1st measurement beams across a skin region to determine treatment region and treatment parameters for treatment beams, before start of treatment for said treatment region.
E6. A method according to E5, interrupting transmissions for at least one treatment beam with 2ndtype measurement beams to determine at least one of i) temperature and ii) degree of ablation (coagulation) of the skin region under treatment, to determine end of treatment for said treatment beams.
E7. A method according to E6, using infrared methods to determine end of transmission for the treatment beams.

Method or system according to all previous claims in combination with optical temperature detection measures.

This invention covers the use of systems described herein. The system can operate in real or approximate real time.

The present invention is not limited to the described apparatus, system or algorithms, thus all devices and the use thereof that are functionally equivalent are included by the scope of the invention. Modifications of the patent claims are within the scope of the invention.

Drawings and figures are to be interpreted illustratively and not in a limiting context. It is further presupposed that all the claims shall be interpreted to cover all generic and specific characteristics of the invention which are described, and that all aspects related to the invention, no matter the specific use of language, shall be included. Thus, the stated references have to be interpreted to be included as part of this invention's basis, methodology, mode of operation and apparatus or system.

1. A system for treating wrinkles and rejuvenating skin on a human body, the system comprising a diagnostic component and a therapeutic component, an ultrasound probe, wherein the diagnostic component and the therapeutic component are connected to the ultrasound probe for diagnosis and therapy, a processor, and a memory, the processor running a program stored in the memory causing the system to perform the following steps:
obtaining an image of a depth of the skin in a region of interest on the skin using the diagnostic component; and
at each target point of a plurality of target points in the region of interest, determining how many ultrasound therapy foci to apply and the depths of each of the ultrasound therapy foci based on the image, and applying the ultrasound therapy foci at each of the depths using the therapeutic component.

2. The system of claim 1, wherein the ultrasound probe comprises one of:
i) a single ultrasound transducer or transducer array that is used by both the diagnostic component and the therapeutic component;
ii) separate transducers or transducer arrays used respectively for the diagnostic component and the therapeutic component, and angled so that diagnostic beams and therapeutic beams overlap in the skin; and
iii) separate transducers or transducer arrays used respectively for the diagnostic component and the therapeutic component, the separate transducers or transducer arrays being mounted in an acoustic stack or an annular structure so that a diagnostic beam axis and a therapeutic beam axis overlap.

3. The system of claim 2, wherein the ultrasound probe transmits diagnostic ultrasound at a diagnostic frequency (DF) and therapeutic ultrasound at a therapeutic frequency (TF), wherein DF≥1.5·TF.

4. The system of claim 1, wherein the processor running the program causes the system to further perform the steps:
determining whether a skin thickness at the each target point is greater than a predetermined minimum thickness based on the image;
if the skin thickness at the each target point is greater than a predetermined defined minimum thickness, then performing the steps of determining and applying at the each target point; and
if the skin thickness at the each target point is not greater than a predetermined minimum thickness, then not performing the steps of determining and applying at the each target point.

5. The system of claim 1, wherein the processor running the program causes the system to further perform, during the step of applying, measuring variations in a tissue parameter at the location of the each of the ultrasound therapy foci using the diagnostic component, and ceasing the step of applying of the each of the ultrasound therapy foci when the variations in tissue parameter meet a predetermined value.

6. The system of claim 1, wherein the image is a 3D image of the depth in the region of interest.

7. The system of claim 1, wherein the image is a 2D image of the depth obtained along a line in the region of interest.

8. The system of claim 7, wherein the processor running the program further causes the system to move the transducer in a direction orthogonal to the line and repeat the steps of obtaining along additional lines to obtain a plurality of 2D images that are combinable to form a 3D image of the region of interest.

9. The system of claim 8, wherein the steps of determining and applying are performed for each of the additional lines.

10. The system of claim 8, wherein the steps of determining and applying are performed for each of the 2D images before a successive one of the 2D images is obtained.

11. The system of claim 8, wherein the transducer moves within the probe in the direction orthogonal to the line to obtain the plurality of 2D images.

12. The system of claim 7, wherein the transducer moves within the probe in the direction orthogonal to the line to obtain a plurality of 2D images.

13. The system of claim 8, further comprising a robotic arm on which the probe is mounted, the robotic arm capable of positioning and orienting the probe, the robotic arm moves the transducer in the direction orthogonal to the line to obtain the 3D image.

14. The system of claim 1, further comprising a holding fixture on which the probe is mounted, the holding fixture capable of maintaining the probe at a fixed position for at least one of obtaining the image and applying the ultrasound therapy foci in a locked position and manually adjustable to change an orientation or position of the probe in an unlocked position.

15. The system of claim 1, further comprising a robotic arm on which the probe is mounted, the robotic arm capable of positioning and orienting the probe, wherein the robotic arm moves the transducer for at least one of obtaining the image and applying the ultrasound therapy foci.

16. The system of claim 1, wherein the diagnostic component is used during the step of applying the ultrasound therapy foci to correct for body movements.

17. The system of claim 5, wherein the variations in the tissue parameter include changes in an elastic stiffness of the tissue.

18. The system of claim 5, wherein the variations in the tissue parameter include changes in an optical property of the tissue.

19. The system of claim 17, wherein the elastic stiffness is measured using an acoustic radiation force (ARF) mode of the diagnostic component to displace the skin.

20. The system of claim 1, wherein the probe includes an acoustic standoff providing acoustic contact to a region of a multi-curved skin surface with low absorption so that a high ultrasound intensity is obtained in a subcutaneous focus region.
